(19) United States
(12) Patent Application Publication
(10) Pub. No.: US 2010/0004523 A1
(43) Pub. Date: Jan. 7, 2010

(54) Active, radiating low frequency implantable sensor and radio tag system

(75) Inventors: Jason August, Toronto (CA); Paul Waterhouse, Selkirk (CA); John K. Stevens, Stratham, NH (US); Michael J. Vandenberg, Ontario (CA); Kenneth Truong, Scarborough (CA)

Correspondence Address: MICHAEL J. BUCHENHORNER, 8540 S.W. 83 STREET, MIAMI, FL, 33143, US

(73) Assignee: Visible Assets, Inc.

(21) Appl. No.: 11/402,413

(22) Filed: Apr. 12, 2006

Related U.S. Application Data
(60) Provisional application No. 60/773,306, filed on Feb. 14, 2006.

(51) Int. Cl. A61B 5/145; A61B 5/0402; G08B 1/08

(52) U.S. Cl. 600/365; 600/509; 340/539.12

(57) ABSTRACT

A low frequency implantable sensor and radio tag system, includes a sensor device which in turn includes: storage storing information including information identifying the device; an apparatus, coupled to the transceiver, for measuring a body condition for transmission to a reader; a transceiver, coupled to the storage, the transceiver operating at a frequency sufficiently low to operate near or within water; and an antenna, coupled to the transceiver, communicating with an external reader.

### CROSS-REFERENCE TO RELATED APPLICATIONS

This Application claims priority from: U.S. Provisional Application 60/773,306, “Active, Radiating Low Frequency Implantable Sensor and Radio Tag System, and U.S. Provisional Application No 60/652,554, “Ultra Low Frequency Tag and System,” Ser. No.10/820,366, “Damage Alert Tag,” U.S. Provisional Application 60/627,984, “Auditable authentication of event histories,” U.S. Provisional Application 60/299,727, “System and Method for Packaging and Delivering a Temperature Sensitive Item,” U.S. Provisional Application 60/595,156, “Tagging and Communication System and Methods Therewith,” U.S. Provisional Application 60/700,886, “The Rubee IV Protocol and Its Use in Visibility Networks,” and U.S. Provisional application Ser. No. 10/832,853, “Low Cost Secure ID And System,” U.S. Provisional Application 60/613,767, “RF tags For Tracking And Locating Travel Bags, and U.S. Provisional Application 60/712,730, “Low Frequency Radio Tag And Encapsulating System.”

### FIELD OF THE INVENTION

The present invention relates to an active low frequency (LF, inductive) radiating radio transceiver tag, and a tunable area antenna system that may be used to create low power, highly efficient implantable sensors for humans.

### BACKGROUND OF THE INVENTION

### RFID Background

Radio Frequency IDentification (RFID) tags and telemetry for implantable devices have a long history and, in recent times, RFID has also played a role (e.g., see US Patent Application 2005/0012617 A1) in implantable devices. RFID has become synonymous with “passive back-scattered transponders.” Passive transponders obtain power and a clock reference via a carrier and communicate by de-tuning an antenna most often with a fixed pre-programmed ID. These tags are designed to replace barcodes and are capable of low power, two-way communications. Much of the patent literature and published literature surrounding these radio tags and RFID tags and implantable sensors uses terminology that has not been well defined and can be confusing.

Many previous patents do not make distinctions discussed herein and for example, several of the early issued patents (e.g., U.S. Pat. Nos. 4,724,427, 4,857,893, 3,739,376, and 4,019,181) do not specify the frequency for the preferred embodiment. The frequency can change the radio tag's ability to operate in harsh environments, near liquids, or conductive materials, as well as the tag's range, power consumption and battery life.

For example, US Patent Application 2005/0012617 discloses orthopedic components with data storage elements, and U.S. Pat. No. 6,687,131 discloses a transponder and injection-molded part and method for manufacturing same, referenced by US Patent Application 2005/0012617. These patents do not specify a frequency or mode of operation for a passive RFID tag implanted within an orthopedic joint. Yet commercial RFID tags would be stopped by any steel and water contained in a tissue (HF reduced by 50% and UHF 100%) and a passive LF tag would have a range of only a few inches.

The first reference to a radio tag in the patent literature was a passive radiating transponder described in U.S. Pat. No. 3,406,391, “Vehicle Identification System,” issued in 1968. The device was designed to track moving vehicles. U.S. Pat. No. 3,406,391 teaches that a carrier signal may be used to communicate to a radio tag in addition to providing power. The tags were powered using microwave frequencies and many sub-carrier frequencies were transmitted to the tag. The radio tag was programmed to pre-select several of the sub-carriers and provided an active re-transmission back when a sub-carrier corresponded to a set of pre-programmed bits in the tag. This multi-frequency approach limited data to about five to eight bits and the range of the device was limited to only a few inches.

U.S. Pat. No. 3,541,257, “Communication Response Unit,” issued in 1970, further taught that a digital address may be transmitted and detected to activate a radio tag. The radio tag may be capable of transmitting and receiving electromagnetic signals with memory, may work within a full addressable network, and has utility in many areas. Many other similar devices were described in the following years (e.g., The Mercury News, RFID Pioneers Discuss Its Origins, Sun, Jul. 18, 2004).

U.S. Pat. No. 3,689,885, “Inductively Coupled Passive Responder and Interrogator Unit Having Multidimensional Electromagnetic Field Capabilities,” issued in 1972, and U.S. Pat. No. 3,859,624, “Inductively Coupled Transmitter-Responder Arrangement,” also issued in 1972, teach that a passive radiating digital radio tag may be powered and activated by induction using low frequencies (50 kHz), and transmit coded data modulated at a higher frequency (450 kHz) back to an integrator. They also show that the clock and 450 kHz-transmitting carrier from the radio tag may be derived from the 50 kHz induction power carrier. The inventors propose use of a ceramic filter to multiply the 50 kHz signal 9 times to get a frequency regenerate for the 450 kHz data out signal. These two patents also teach that steel and other conductive metals may de-tune the antennas and degrade performance. The ceramic filter required to increase the frequency from 50 kHz to a higher frequency is, however, an expensive large external component, and phase locked loops or other methods commonly used to multiply a frequency would consume considerable power. These tags use the low frequency “power channel” to power the tag, serve as the time base for the tag, and finally as the trigger for the tag to transmit its ID. Thus, the power channel contains a single bit of on/off information.

U.S. Pat. No. 3,713,148, “Transponder Apparatus and System,” issued in 1973, teaches that the carrier to the transponder may also transmit digital data and that the interrogation device (data input) may also be used to power the transponder. This patent also teaches that non-volatile memory may be added to store data that might be received and to track things like use and costs for tolls. The inventors do not specify or provide details on frequency or antenna configurations.

All devices referenced above rely on the antenna in radiating transceiver mode, where the power from the radio tag is actually “pumped” into a tuned circuit that includes a radiating antenna, which in turn produces an electromagnetic signal that can be detected at a distance by an interrogator.

U.S. Pat. No. 3,427,614, “Wireless And Radioless (Nonradiant) Telemetry System For Monitoring Conditions,” issued in 1969, was the first to teach that the radio tag antenna may communicate simply by de-tuning the antenna rather than radiating power through the tuned antenna. The change in tuned frequency may be detected by a base station generating a carrier. This non-radiating mode reduces the power required to operate a tag and puts the detection burden on the base station. In effect, the radio tag's antenna becomes part of a tuned circuit created by the combination of the base station and a carrier. Any change in the radio tag's tuned frequency by any means can be detected by the base station's tuned carrier circuit. This is also often referred to as a back-scattered mode and is the basis for most modern RFID radio tags.

Many Electronic Article Surveillance (EAS) systems also function using this back-scattered non-radiating mode (see U.S. Pat. Nos. 4,774,504, 3,500,373, 5,103,234), and most are also inductive frequencies. Many other telemetry systems in widespread use for pacemakers, implantable devices, and sensors in rotating centrifuges (see U.S. Pat. No. 3,713,124 for “Temperature Telemetering Apparatus”) also make use of this back-scattered mode to reduce power consumption. U.S. Pat. No. 4,361,153 “Implant Telemetry System” teaches that low frequencies (myriametric) can transmit through conductive materials and work in harsh environments. Most of these implantable devices also use the back-scattered communication mode for communication to conserve battery power.

Accordingly, more recent and modern RFID tags are passive, back-scattered transponder tags and have an antenna consisting of a wire coil or an antenna coil etched or silk screened onto a PC board (e.g., see U.S. Pat. No. 4,857,893, “Single chip Transponder Device,” 1989; U.S. Pat. No. 5,682,143, “Radio Frequency Identification Tag”). These tags use a carrier that is reflected back from the tag. The carrier is used by the tag for four functions:

The carrier contains the incoming digital data stream signal, in many cases the carrier only performs the logical function to turn the tag on/off and activate the transmission of its ID. In other cases, the data may be a digital instruction. The carrier serves as the tag's power source. The tag receives a carrier signal from a base station and uses the rectified carrier signal to provide power to the integrated circuitry and logic on the tag. The carrier serves as a clock and time base to drive the logic and circuitry within the integrated circuit. In some cases, the carrier signal is divided to produce a lower clock speed.

The carrier may also serve as a frequency and phase reference for radio communications and signal processing. The tag can use one coil to receive a carrier at a precise frequency and phase reference for the circuitry within the radio tag for communications back through a second coil to the reader/writer, making accurate signal processing possible (see U.S. Pat. No. 4,879,756 for “Radio Broadcast Communication Systems”).

Thus, the main advantage of a passive back-scattered transponder is that it eliminates the battery as well as a crystal in LF tags. HF and UHF tags are unable to use the carrier as a time base because it would require high speed chips and power consumption would be too high. It is therefore generally assumed that a passive back-scattered transponder tag is less costly than an active or transceiver tag since it has fewer components and is less complex.

These modern non-radiating transponder back-scattered RFID tags typically operate at frequencies within the Part 15 rules of the FCC (Federal Communication Commission), between 10 kHz to 500 kHz (low frequency, LF, or ultra low frequency, ULF), 13.56 MHz (high frequency, HF), or 433 MHz (MHF) and 868/915 MHz or 2.2 GHz (ultra high frequency, UHF). The higher frequencies are typically chosen because they provide high bandwidth for communications, on a high speed conveyor for example, or where many thousands of tags must be read rapidly. In addition, it is generally believed that the higher frequencies are more efficient for transmission of signals and require much smaller antennas for optimal transmission. (It may be noted that a self-resonated antenna for 915 MHz can have a diameter as small as 0.5 cm and may have a range of tens of feet.)

However, the major disadvantage of the back-scattered mode radio tag is that it has limited power, limited range, and is susceptible to noise and reflections over a radiating active device. This is largely because the passive tag requires a minimum of one volt on its antenna to power the chip, not because of loss of communication signal. As a result, many back-scattered tags do not work reliably in harsh environments and require a directional “line of site” antenna.

One method to extend the range of a passive back-scattered tag has been to add a thin, flat battery to the back-scattered tag so the power drop on the antenna is not the critical range limiting factor. However, since all of these tags use high frequencies, the tags must continue to operate in back-scattered mode to conserve battery life. The power consumed by any electronic circuit tends to be related to the frequency of operation. Thus, if a chip were to use an industry standard 280 Mah-capacity CR2525 Li cell (size of a quarter), we would expect, based solely on operating frequency, battery life to be:

Assumes 280 MaHr Li Battery

| | | Power (uAHr) | Predicted | |
|---|---|---|---|---|
| Freq. | | Current (uA) | Life | Units |
| 128 | kHz | 1 | 31.00 | Years |
| 13.56 | MHz | 102 | 3.78 | Months |
| 915 | MHz | 7,031 | 1.66 | Days |

Thus, most recent active RFID tags that may have a battery to power the tag circuitry are active tags and devices operating in the 13.56 MHz to 2.3 GHz frequency range, and also work as back-scattered transponders (U.S. Pat. No. 6,700,491, “Radio Frequency Identification Tag With Thin-Film Battery For Antenna,” 2004; also see US Patent Application 2004/0217865, “RFID Tag,” for a detailed overview of issues). Because these tags are active back-scattered transponders, they cannot work in an on-demand peer-to-peer network setting, or require line of sight antennas that provide a carrier that “illuminates” an area or zone or an array of carrier beacons.

Active radiating transceiver tags in the high frequency range (433 MHz) that can provide an on-demand peer-to-peer network of tags are available (e.g., SaviTag ST-654, U.S. Pat. No. 5,485,166, “Efficient Electrically Small Loop Antenna With A Planar Base Element,” 1996) with full visibility systems described above (see U.S. Pat. No. 5,686,902, U.S. Pat. No. 6,900,731). These tags do provide full functionality and so-called Real-Time Visibility, but they are expensive (over $100.00 US) and large (videotape-size, 6.25×2.125×1.125 inches) because of the power issues described above. They must also use replaceable batteries since even with a 1.5-inch by 6-inch Li battery, these tags are only capable of 2,500 reads and writes.

It is also generally assumed that HF or UHF passive back-scattered transponder radio tags will have a lower cost to manufacture than an LF passive back-scattered transponder because of the antenna. An HF or UHF tag can obtain a high Q, 1/10 wavelength antenna by etching or conductive silver silk screening the antenna geometry onto a flexi-circuit. An LF (30 to 300 KHz) or ULF (300-3000 Hz) antenna cannot use either because the Q will be too low due to high resistance of the traces or silver paste. Therefore, LF and ULF tags must use wound coils made of copper.

Thus, in summary, a passive transponder tag has the potential to lower cost by eliminating the need for a battery as well as an internal frequency reference means. An active back-scattered transponder tag eliminates the extra cost of crystal while also providing for enhanced amplification of signals over a passive back-scattered transponder and enhanced range. In addition, it is also possible to use carrier reference to provide enhanced anti-collision methods to make it viable to read many tags within a carrier field (U.S. Pat. Nos. 6,297,734, U.S. Pat. No. 6,566,997, U.S. Pat. No. 5,995,019, and U.S. Pat. No. 5,591,951). Finally, active radiating transceiver tags require large batteries and are expensive, perhaps costing up to hundreds of dollars.

The prior art has assumed low frequency tags are slow, short-ranged, and too costly. For example, both U.S. Pat. No. 5,012,236 and U.S. Pat. No. 5,686,902 discuss the short range issues associated with magnetic induction and low frequency tags. Because of the many apparent disadvantages of ULF and LF, the RFID frequencies are now recommended by many commercial (see Item-Level Visibility In the Pharmaceutical Supply Chain: A Comparison of HF, UHF, and RFID Technologies, July 2004, Texas Instruments, Phillips Semiconductors, and TagSys Inc.) and government organizations (see Radio Frequency Identification Feasibility Studies and Pilot, FDA Compliance Policy HFC-230, Sec 400.210, November, 2004, recommend use of LF, HF or UHF), and standards associations (EPCglobal, web page tag specifications, January 2005, note LF and ULF are excluded) do not mention or discuss the use of ULF as an option in many important retail applications. Many of the commercial organizations recommending these higher frequencies believe that passive and active radio tags in low frequencies are not suitable for any of these applications for reasons given above.

In addition, several commercial companies actually manufacture both ULF and LF radio tags (e.g., Texas Instruments and Philips Semiconductor, see Item-Level Visibility In the Pharmaceutical Supply Chain, A Comparison of HF, UHF, RFID Technologies, July 2004, Texas Instruments, Phillips Semiconductors, and TagSys Inc.), yet only recommend the use of 13.56 MHz or higher, again because of the perceived disadvantage of ULF and LF mentioned above, and the many perceived advantages of HF, and UHF.

Current LF radiating active radio tags have not been considered for use in many modern applications. ULF is believed to have very short range since it uses largely inductive or magnetic radiance that drops off 1/d³, while far field HF and UHF drops off 1/d, where d is the distance from the source. Thus, the inductive or magnetic radiance mode of transmission will theoretically limit the distance of transmission, and that has been one of the major justifications for use of HF and UHF passive radio tags in many applications.

The transmission speed is inherently slow using ULF as compared to HF and UHF since the tag must communicate with low baud rates because of the low transmission carrier frequency. Many sources of noise exist at these ULF frequencies from electronic devices, motors, fluorescent ballasts, computer systems, and power cables. Thus, ULF is often thought to be inherently more susceptible to noise. Radio tags in this frequency range are considered more expensive since they require a wound coil antenna because of the requirement for many turns to achieve optimal electrical properties (maximum Q). In contrast, HF and UHF tags can use antennas etched directly on a printed circuit board. ULF would also have even more serious distance limitations with such an antenna. Current networking methods used by high frequency tags, as used in HF and UHF, are impractical due to such low bandwidth of ULF tags described above.

Low frequency, active radiating transceiver tags are especially useful for visibility and for tracking objects with large area loop antennas over other more expensive active radiating transponder HF/UHF tags (e.g., Savi ST-654). These LF tags will function in harsh environments, near water and steel, and may have full two-way digital communications protocol, digital static memory and optional processing ability, sensors with memory, and ranges of up to 100 feet. The active radiating transceiver tags can be far less costly than other active transceiver tags (many under one dollar), and often less costly than passive back-scattered transponder RF-ID tags, especially those that require memory and make use of EEPROM. These low frequency radiating transceiver tags also provide a high level of security since they have an on-board crystal that can provide a date-time stamp, making full AES encryption and one-time based pads possible. Finally, in most cases, LF active radiant transponder tags have a battery life of 10-15 years using inexpensive CR2525 Li batteries with 1,000,000 or more transmissions.

These LF radiating transceiver tags may be used in a variety of applications; however, their intended use is within visibility networks for tracking assets in warehouses and moving vehicles, and they overcome many of the disadvantages of a passive back-scattered transponder tag system (U.S. Pat. No. 6,738,628, “Electronic Physical Asset Tracking”). The tags may also be used for visibility networks for airline bags, evidence tracking, and livestock tracking, or in retail stores for tracking products.

U.S. Pat. No. 4,361,153, “Implant Telemetry System,” issued in 1983, U.S. Pat. No. 4,494,545, “Implant Telemetry System,” issued in 1985, and U.S. Pat. No. 4,571,589, “Biomedical Implant With High Speed, Low Power Two-Way Telemetry,” issued in 1986, review much of the prior art based on use of magnets and reed switches to program pacemakers and other medical devices. U.S. Pat. Nos. 4,361,153; 4,494,545 and 4,571,589 teach that a passive backscattered low frequency (myriametric frequencies ) system can be used to program a pacemaker with no power requirement from the pacemaker itself, thereby minimizing power required in the pacemaker and maximizing battery life. U.S. Pat. No. 4,361,153 also reviews that it is possible to use the same coils used to power the tags with low frequency carriers to charge the batteries in the pacemakers. Theses tags all work in transponder mode and communicate by de-tuning the antenna.

### Implantable Background

Implantable telemetry systems have relied either on high frequency or low frequency backscattered modes of operation, and in many cases wired or short range systems have been proposed.

U.S. Pat. No. 4,361,153, “Implant Telemetry System” (1982) emphasizes issues associated with no power and limited power budget. U.S. Pat. No. 6,122,536, “Implantable Sensor And System For Measurement and Control Of Blood Constituent Levels,” issued in 2000, teaches how many sensors may be created to monitor blood flow, oxygen, and its clinical value. It assumes, however, that the sensor will be monitored via wires or connections though the patient, or via contact with the skin using Infrared Radiation (IR).

U.S. Provisional Application 60/652,554, “Ultra Low Frequency Tag and System,” U.S. application Ser. No. 10/820,366, “Damage Alert Tag,” U.S. Provisional Application 60/627,984, “Auditable Authentication Of Event Histories,” 60/299,727.

U.S. Pat. No. 3,672,352, “Implantable Bio-Data Monitoring Method and Apparatus,” uses IR through the skin. U.S. Pat. No. 6,895,281, “Inductive Coil Apparatus For Bio-Medical Telemetry,” uses short range inductive coils.

U.S. Pat. No. 6,167,312, “Telemetry System For Implantable Medical Devices” (issued 2000), makes use of a 400 Mhz UHF system. U.S. Pat. No. 6,917,833, “Omni Directional Antenna For Wireless Communication With Implanted Medical Devices” (issued 2005), makes use of a 27 Mhz HF system. Finally, U.S. Pat. No. 6,847,844, “Method of Data Communication With Implanted Device And Associated Apparatus” (issued 2005), teach use of current flowing through tissue as a replacement for direct wires connected through the torso, or RF based on passive or battery based tags. Therefore, there is a need for a sensor system that overcomes the foregoing shortcomings the prior art.

### SUMMARY OF THE INVENTION

Briefly, according to an embodiment of the invention, a version of a low frequency (LF) active transceiver tag with sensors can be used as an implantable device. These tags have the advantage of a long battery life (e.g., ten years) and can function in a full peer-to-peer network with small antennae attached to the person, or a belt or as a loop in a room. The LF implantable transceiver devices may be used to sense temperature, heart rate, glucose, and any other parameters that can be sensed and not consume excessive power during detection.

According to another embodiment of the invention the sensor has the ability to operate near and around steel or liquids. This is particularly useful for a sensor used in orthopedic implants that are made of high grade stainless steel. The body is made up mostly of liquids so any telemetric system to be optimal must be immune to both effects from steel and water.

According to another embodiment of the invention an optional fixed reader, that can be worn by the patient, has the ability to read the sensor. This can be used to indicate real-time status of the sensors and indicate a fault condition.

According to another embodiment of the invention the sensor has the ability to read LF transceivers using loop antennas within a room. Thus, the data from the implant can be read without a fixed remote reader. It is possible to place an antenna in a room and read the implant anywhere within the area of the room without patient cooperation or a change in behavior. The reader can be connected to a wide area network, such as the Internet, or a local computer directly and data maybe captured and maintained at a remote site with no patient training or special equipment. Thus the patient's bedroom or the hospital room may have a web-enabled reader.

According to another embodiment of the invention the sensor can measure many other physical parameters such as the number of flexes of a joint, total angle of flexion, and statistics associated with walking can be captured. This makes it possible to detect at a low cost any problem with the implant at the earliest possible time.

According to another embodiment of the invention the sensor has the ability to be adapted to work with glucose detectors, radiation monitors, EKG monitors, and any other physiological parameter that can be detected with a sensor, with the same benefits of long range, long battery life and low cost.

### BRIEF DESCRIPTION OF THE DRAWINGS

FIG. 1 shows a sensor implanted next to a knee implant capable of monitoring temperature, strain in the joint, angle of the joint and acceleration.

FIG. 2 shows a possible embodiment wherein an antenna might be externally attached to the outside of the patient to pick up the signals from the implant.

FIG. 3 shows a second possible embodiment comprising a small antenna

FIG. 4 shows examples of actual implant prototypes and monitor antennas.

FIG. 5 shows data comparing coils in FIG. 4 in open air and implantable coil in water.

FIG. 6 shows a graph summarizing the data shown in FIG. 5.

FIG. 7 shows a second embodiment wherein a reader that has been web enabled is attached to an antenna.

FIG. 8 shows tests that were carried out by taping the prototype implantable device to the side of a knee

FIG. 9 illustrates test conditions for the device shown in FIG. 8.

FIG. 10 shows a graph of signal versus time.

FIG. 11 is a third embodiment using a large loop antenna placed around the room.

FIG. 12 The implant was held 3′ off the floor and two tests were performed.

FIG. 13 shows raw data reflecting the signal versus time for test A and test B.

FIG. 14 shows a test using a coil and reader identical to test in FIGS. 9-10.

FIG. 15 shows an experimental arrangement.

FIG. 16 shows the raw data with an open coil (no steel) and the bone rasp.

FIG. 17 shows summary graphs that show that the steel does decrease the range by about 20-30% however it is acceptable at near 6 feet from the antenna.

FIG. 18 shows many other applications that exist for LF radiating transceivers as an implantable device.

FIGS. 19-21 show block diagrams of a typical implantable transceiver.

### DETAILED DESCRIPTION

We provide a glossary of terms and concepts used within this patent disclosure:

Radio Tag: Any telemetry system that communicates via magnetic (inductive communications) or electric radio communications to a base station or reader, or to another radio tag.

Passive Radio Tag: A radio tag that does not contain a battery.

Active Radio Tag: A radio tag that does contain a battery.

Transponder: A radio tag that requires a carrier from an integrator or base station to activate transmission or another function. The carrier is typically used to provide both power and a time-base clock.

Non-Radiating Transponder: A radio tag that may be active or passive and communicates via de-tuning or changing the tuned circuit of an antenna or coil; does not induce power into a transmitting antenna or coil.

Radiating Transponder: A radio tag or transponder that may be an active or passive tag, but communicates to the base station or interrogator by transmitting a radiated detectable electromagnetic signal by way of an antenna. The radio tag induces power into an antenna for its data transmission.

Back-Scattered Transponder: A radio tag that is identical to a non-radiating transponder; communicates by, de-tuning an antenna and does not induce or radiate power in the antenna.

Transceiver: A radiating radio tag that actively receives digital data and actively transmits data by providing power to an antenna; may be active or passive.

Passive Transceiver: A radiating radio tag that actively receives and transmits digital data by providing power to an antenna, but does not have a battery and in most cases does not have a crystal or other time base source.

Active Transceiver: A radiating radio tag that actively receives digital data and actively transmits data by providing power to an antenna, and has a battery and in most cases a crystal or other internal time base source.

Inductive Mode: Uses low frequencies, 3-30 kHz VLF or the Myriametric frequency range, 30-300 kHz LF the Kilometric range, with some in the 300-3000 kHz MF or Hectometric range (usually under 450 kHz). Since the wavelength is so long at these low frequencies, over 99% of the radiated energy is magnetic, as opposed to a radiated electric field. Antennas are significantly (10 to 1000 times) smaller than ¼ wavelength or 1/10 wavelength, which would be required to efficiently radiate an electrical field.

Electromagnetic Mode: As opposed to the inductive mode radiation above, 20 the electromagnetic mode uses frequencies above 3000 kHz in the Hectometric range, typically 8-900 MHz, where the majority of the radiated energy generated or detected may come from the electric field, and a ¼ or 1/10 wavelength antenna or design is often possible and utilized. The majority of radiated and detected energy is an electric field.

These implantable sensors can be small (0.75 inch×1 inch×0.25 inch) yet have a range of many feet, with battery life of over ten years using one or two size Li batteries which are about the size of an American quarter-dollar coin. The tags may be read by a small, low power “belt reader,” worn by a patient, or by a LF area reader placed anywhere within a room. Tags for example can be used to monitor joint temperature, joint stress, joint angles and use, cardiac rhythms, glucose, temperature, pH, radiation dose.

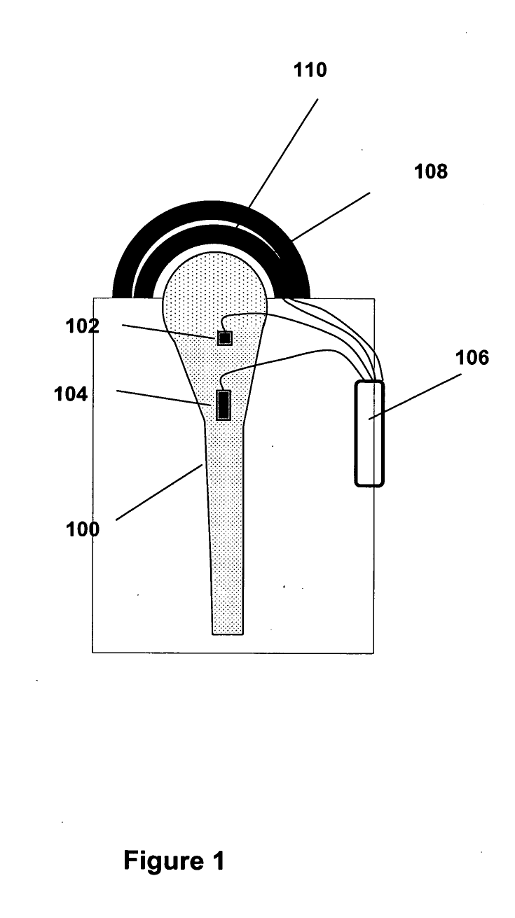

FIG. 1 shows a memory device 106 implanted next to a knee implant capable of monitoring temperature via sensor 108, strain in the joint via sensor 110, angle of the joint via sensor 104 and acceleration via sensor 102. These data may be stored in static memory as a data log and harvested once a day, or may be stored as a histogram in the static memory.

According to another embodiment of the invention an optional fixed reader 102, that can be worn by the patient, has the ability to read the sensor. This can be used to indicate real-time status of the sensors and indicate a fault condition. For example, it has been shown that one major cause of failure of orthopedic implants is a rise in temperature of the joint because of friction between the two surfaces. (The effect of frictional heating and forced cooling on the serum lubricant and wear of Liao Y S, McKellop H, Lu Z, Campbell P, Benya P.; UHMW Polyethylene Cups Against Cobalt-Chromium And Zirconia Balls; Biomaterials. Aug. 24, 2003 (18):3047-59.) This in turn heats the synovial fluid, decreasing lubrication, thus causing further increases in temperature. The ability for the patient to monitor temperature remotely and have an alarm indicating that the knee is over-heating could help prevent this and extend the life of the implant.

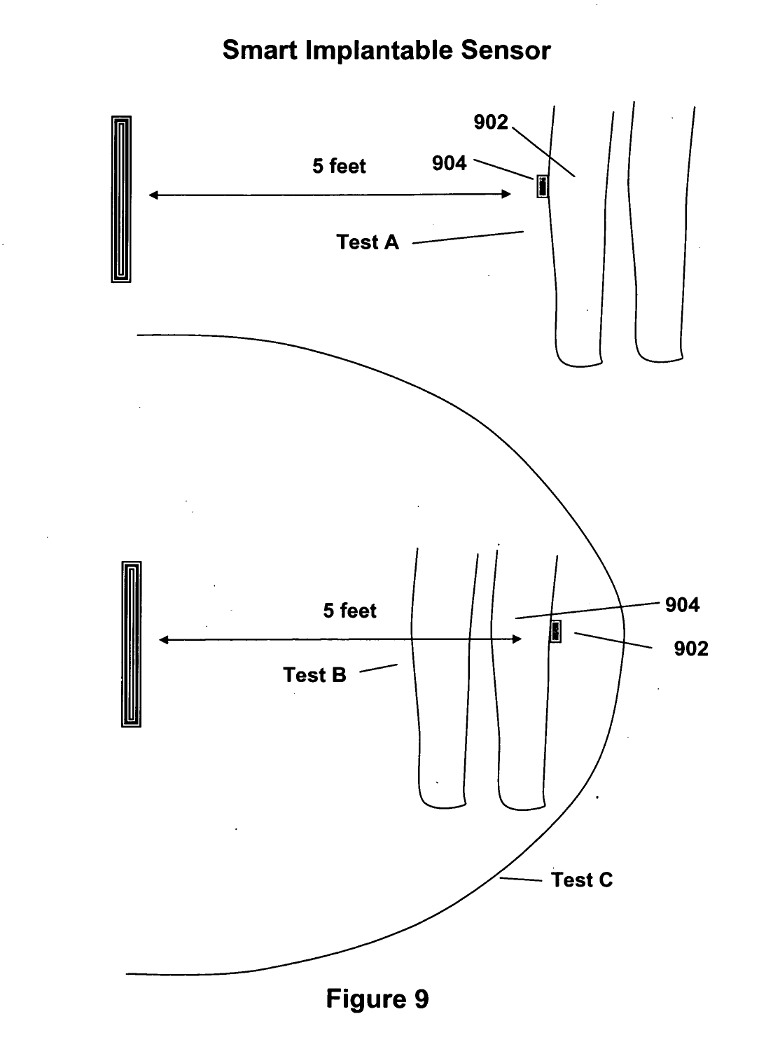

There are advantages of using a ULF system in a knee versus the prior art. These active LF tags may use amplitude modulation, or in some cases, phase modulation, and can have ranges of many tens of feet up to one hundred feet with the use of a loop antenna (see FIGS. 16, 9, 10, 11). The active tags include a battery, a chip and a crystal. As stated above, most often the total cost for such a tag can be less than HF and ULF passive transponder tags, especially if the transponder includes EEPROM, and has a longer range. In cases where the transponder tags use EEPROM, the low frequency active transceiver tag can actually be faster since it uses RAM for storage and write times for EEPROM are quite long. Finally, because these new active transceiver tags use induction as the primary communication mode, and induction works optimally at low frequencies, LF tags are immune to nulls often found near steel and liquids with HF and UHF tags.

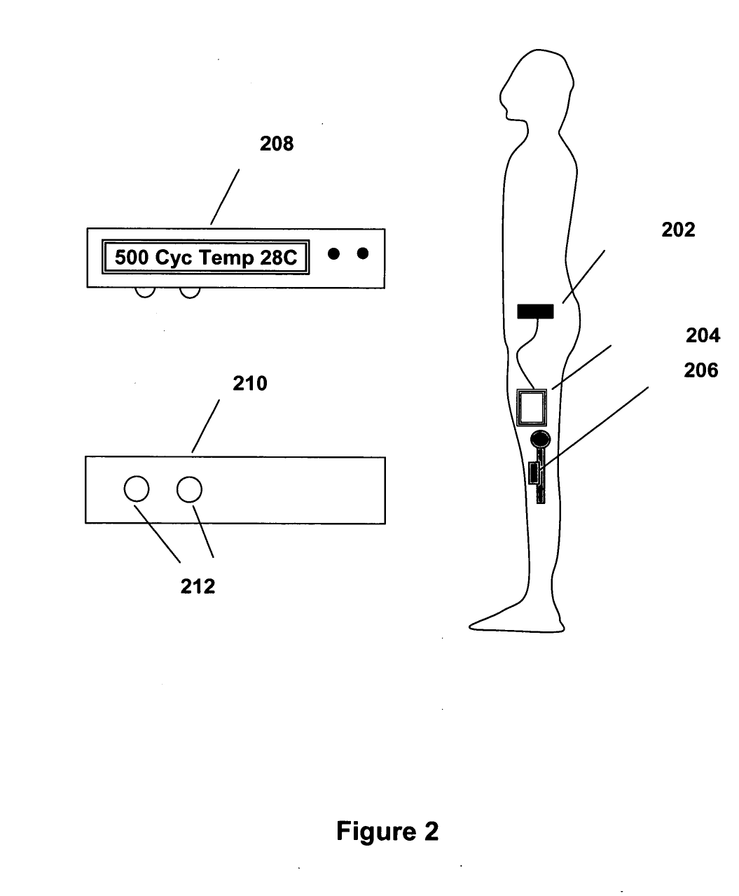

FIG. 2 shows one possible embodiment wherein an antenna 204 is attached to the outside of the patient to pick up the signals from the implant 206. The implant is attached just under the patient's knee. The box or monitor (or reader) 202 may be attached to a belt with a small display 208 on top to indicate status and 210 with optional buttons 212 on the side to control operation.

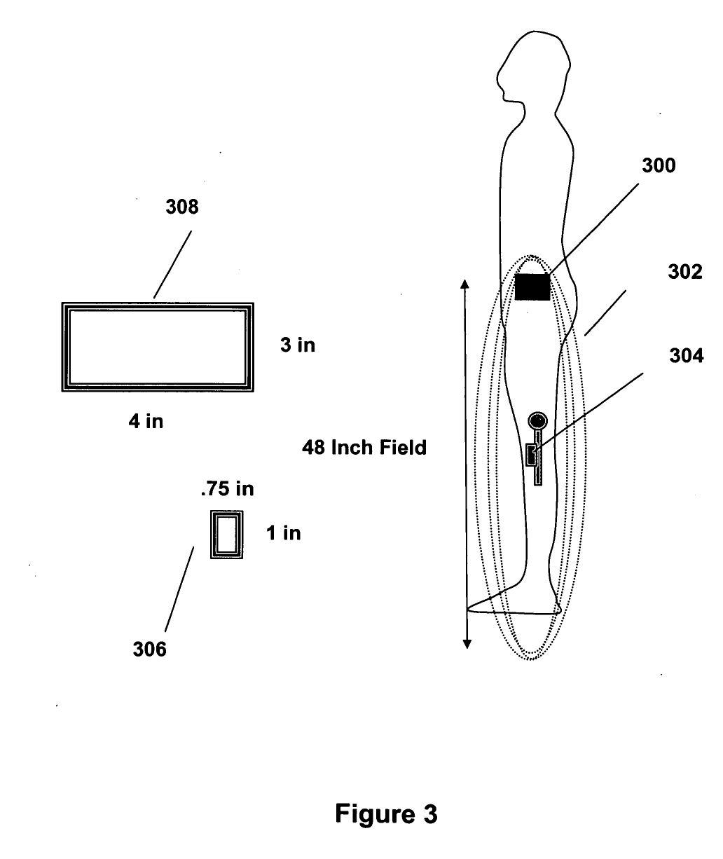

FIG. 3 shows another possible embodiment wherein a small antenna 308 (e.g., 3″×4″) is placed on the monitor 300 itself. This antenna may be optionally in the same plane as the coil 306 in the implant 304. In actual tests, if the coil 306 in the implant has a size of 0.75×0.5 inches, the range will be over 4′. If the implant coil 306 is non optimally oriented, the range may be reduced in the worst case to two to three feet. This arrangement will provide a low cost long battery life monitor and a low cost long battery life implant. A resonant impedance modulated transponder in the implant is used to modulate the phase of a relatively high energy reflected magnetic carrier imposed from outside of the body.

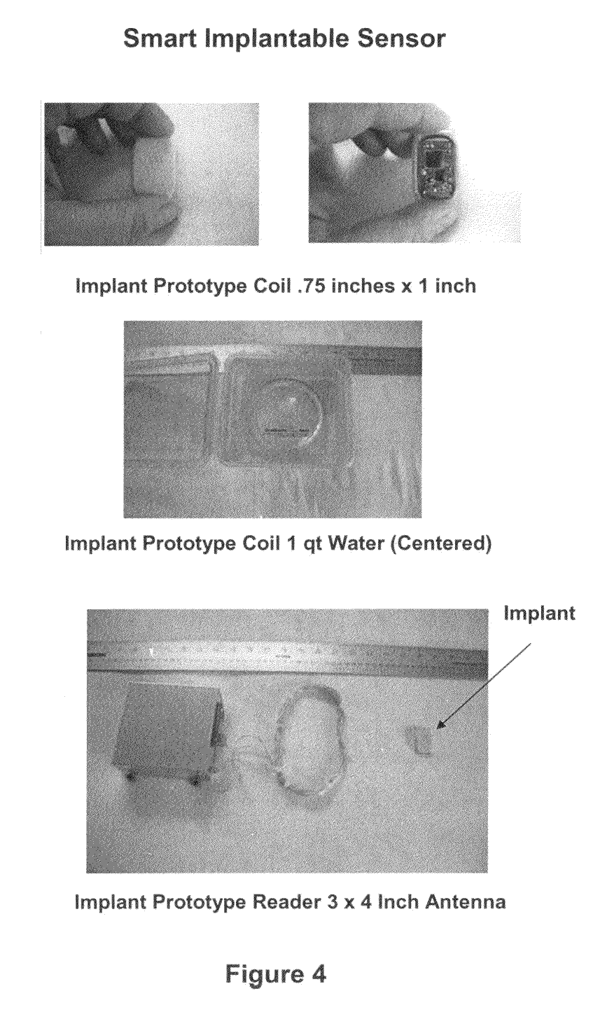

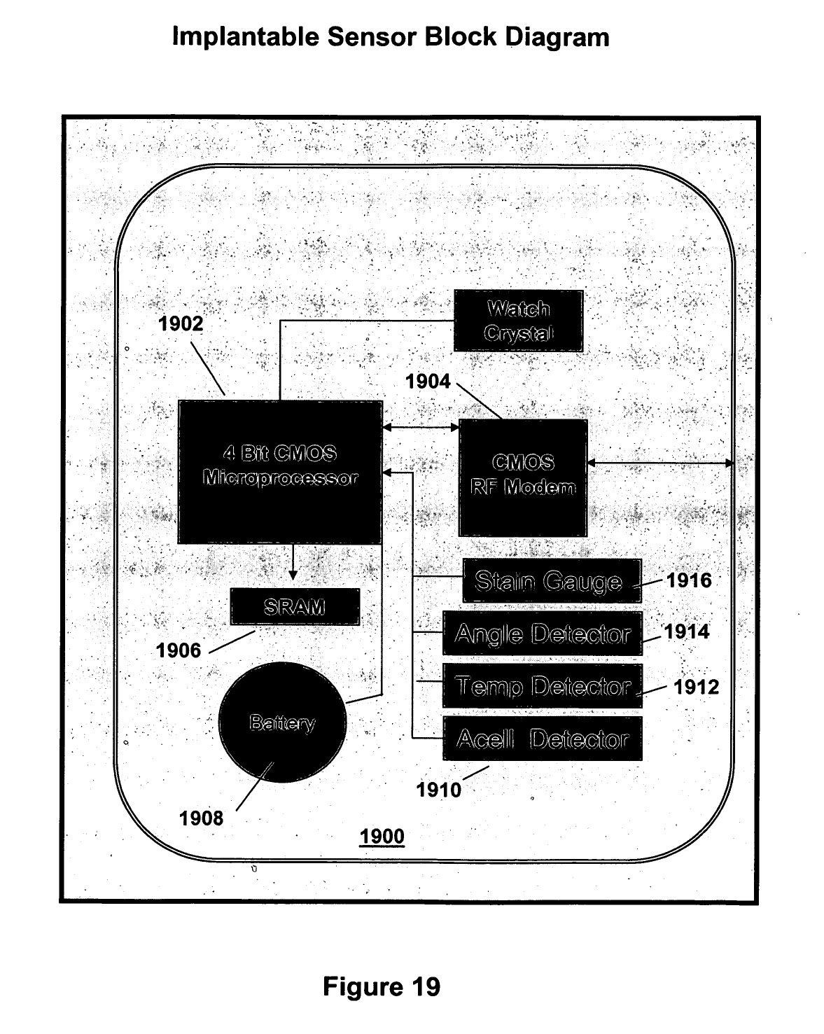

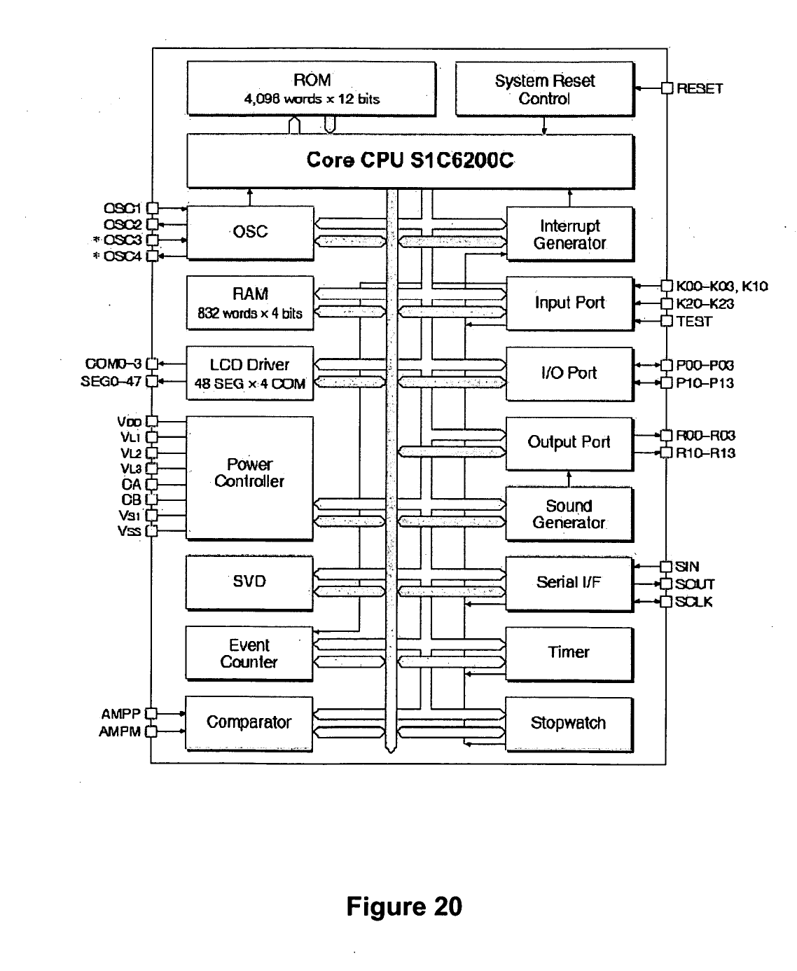

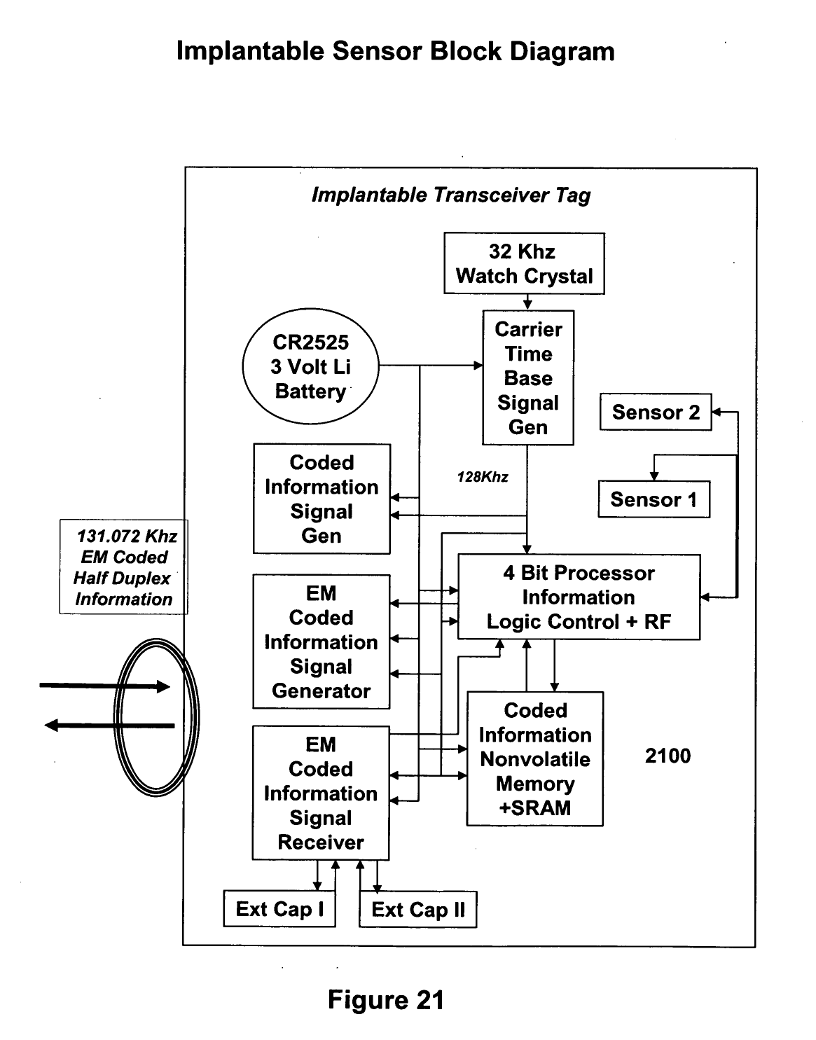

FIG. 4 shows examples of actual implant prototypes and monitor antennas. The implant was placed in a box with a quart of water and held in the middle of the water as a test. The range of the 3×4 inch coil 308 and the 0.75×0.5 inch coil 306 was measured both in water and out of water. The small coil consisted of a circuit shown in FIGS. 19-21 and have a battery life of over ten years. The implantable device operates at 132 Khz and is a full on-demand peer-to-peer, radiating transceiver. The base station was tuned to the 3×4 inch antenna 308.

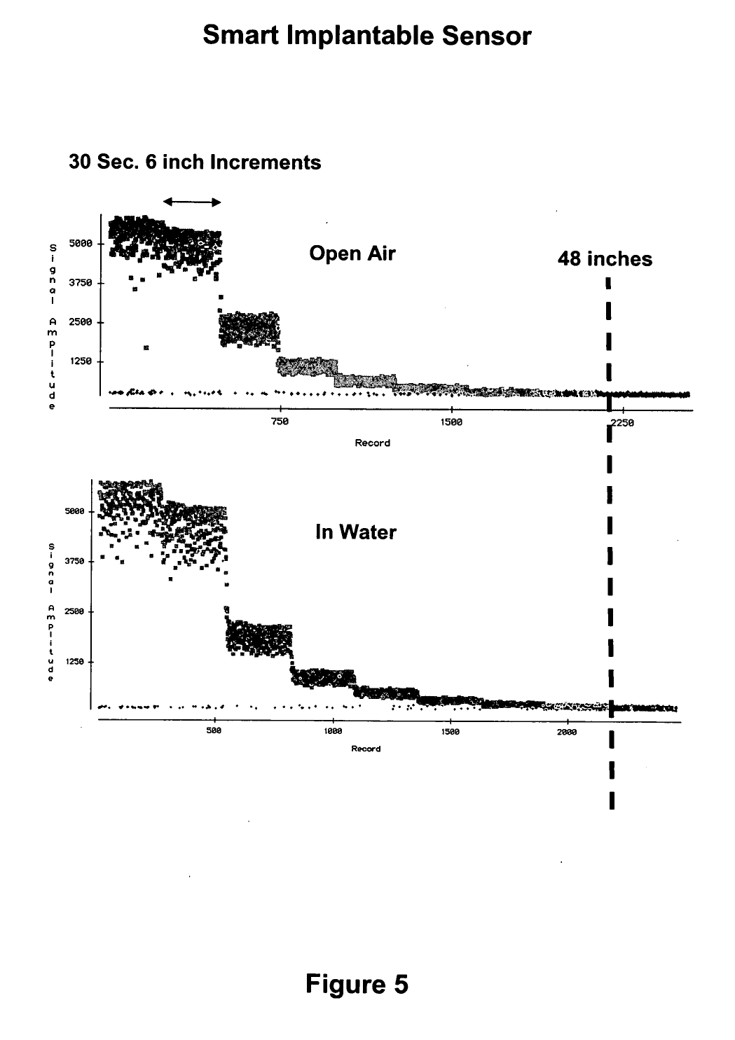

FIG. 5 shows data comparing coils in FIG. 4 in open air and implantable coil in water. No significant difference could be found. This demonstrates that the LF transceiver mode is not affected by liquids.

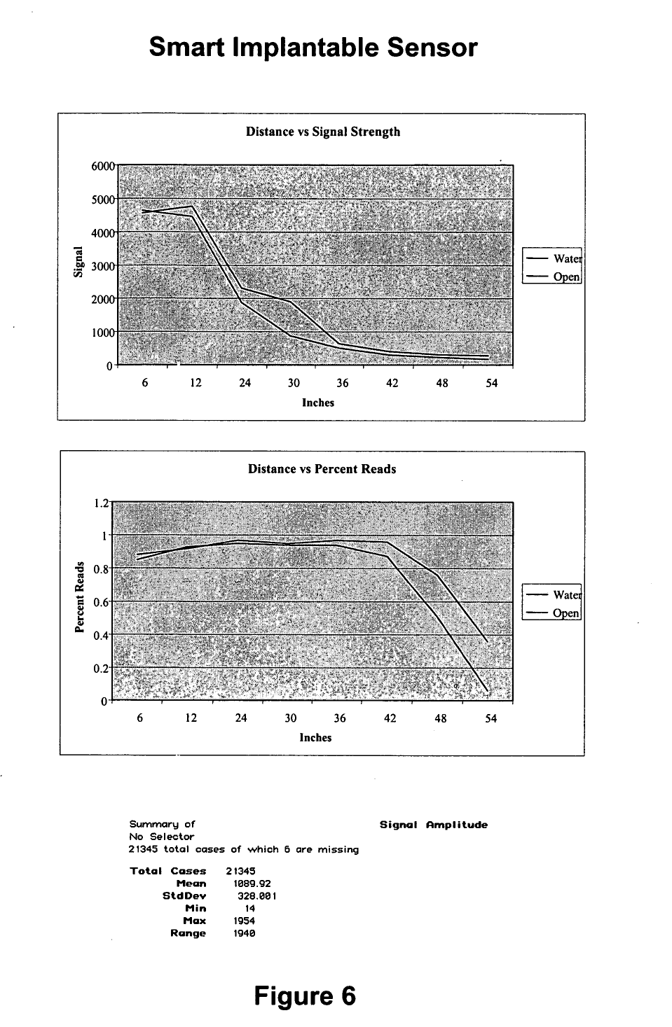

FIG. 6 shows a graph summarizing the data shown in FIG. 5. Again, it shows no significant loss in signal strength as a result of water. The lower graph shows errors associated with reading and writing to the memory of the implantable device. Both confirm no significant changes with liquids. This is not true for any frequency above 1 MHz. Radio signals in the 13.56 MHz range have losses of over 50% in signal strength as a result of water, and anything over 30 MHz have losses of 99%. In addition, as the frequency goes up the power required to operate the implant also increases, so battery life is reduced.

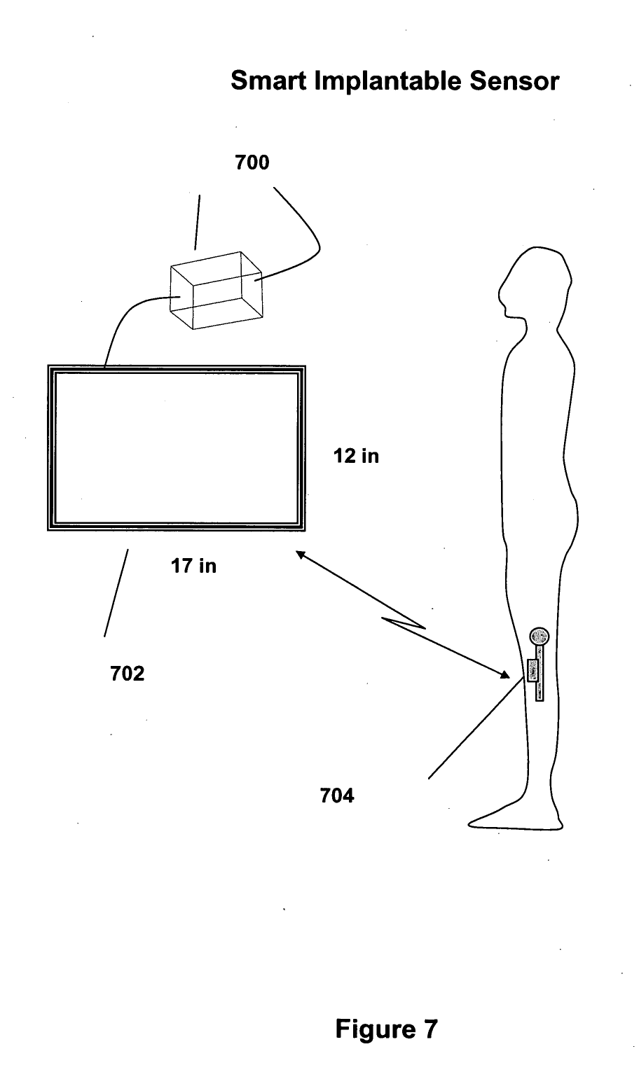

FIG. 7 shows another embodiment wherein a reader 700 that has been web enabled is attached to an antenna 702 about 12×17 inches and placed in a room where a patient wearing an implantable device 704 is located. In this case the patient does not have to wear a monitor and the implantable device may be read from a distance without help or cooperation form the patient.

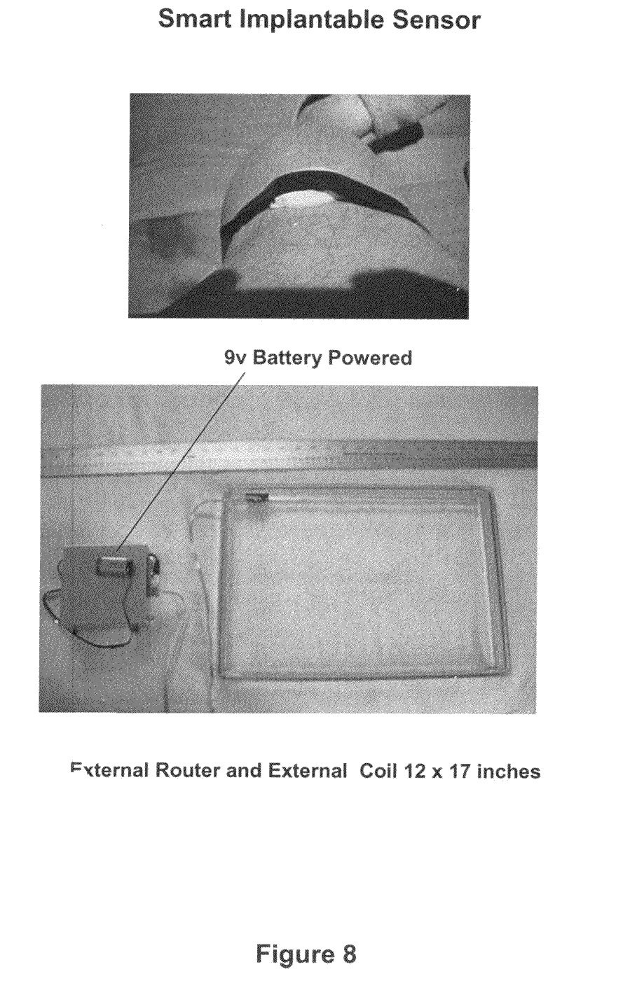

FIG. 8 shows tests that were carried out by taping the prototype implantable device to the side of a knee The readers are also low power devices and as illustrated here can operate for 8 hours on Li 9 volt battery.

FIG. 9 illustrates test conditions for the devices shown in FIG. 7. The antenna 900 was placed about five feet from the test knee 902 and it was tested with the tag 904 on the same side as the antenna 900 (test A) and with the knee 902 and tag 904 on the opposite side of the antenna (test B) so the signal had to go through the test subject's legs to work properly. A third test (C) was also carried out where the test subject walked randomly around a circle about six feet away from the antenna.

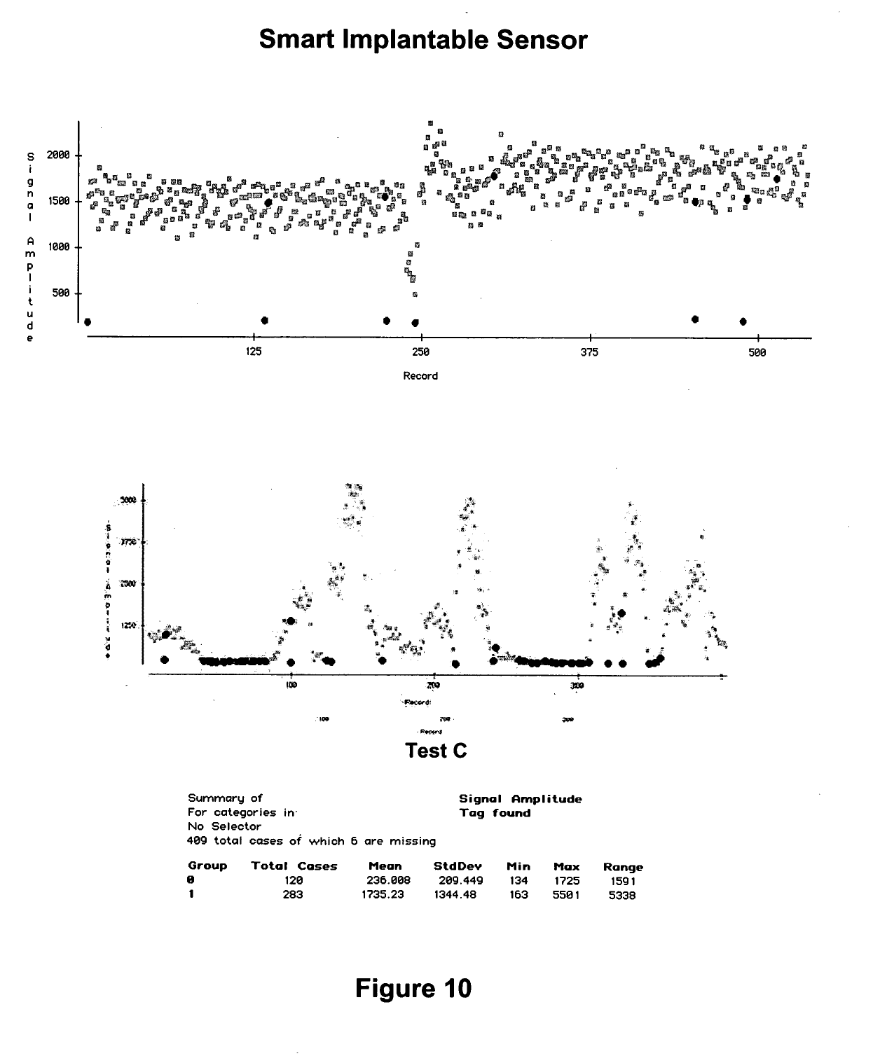

FIG. 10 shows a Graph of signal versus time—lighter dots are a positive CRC and read and darker dots are a bad CRC and error. The raw data shows no difference could be detected between A and B. The C test shows errors in some areas but as the subject walks around many positions provide strong error free reads.

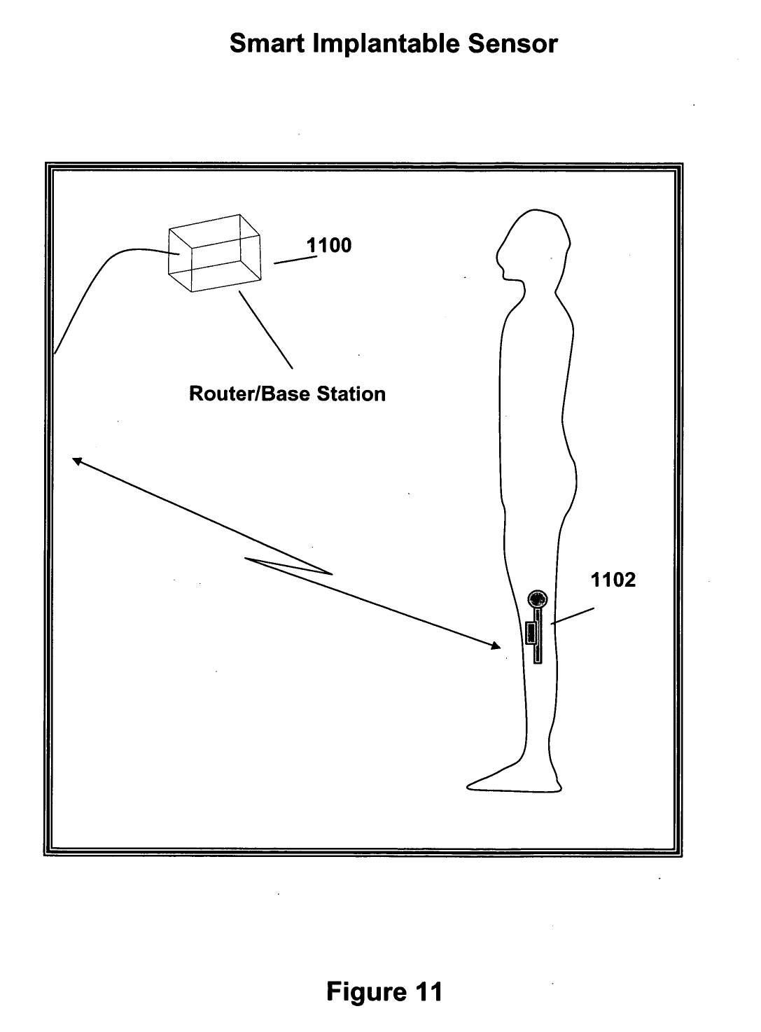

FIG. 11 shows yet another embodiment using a large loop antenna (not shown) placed around the room where the patient wearing the implant 1102 is located. The large loop antenna is connected to a reader in a router/base station 1100. In this case the reader was optimally tuned for this specific loop of about 8′ by 16′ and the loop was draped on the floor around the room. The router/base station 1100 connects the room to a network to allow for remote monitoring.

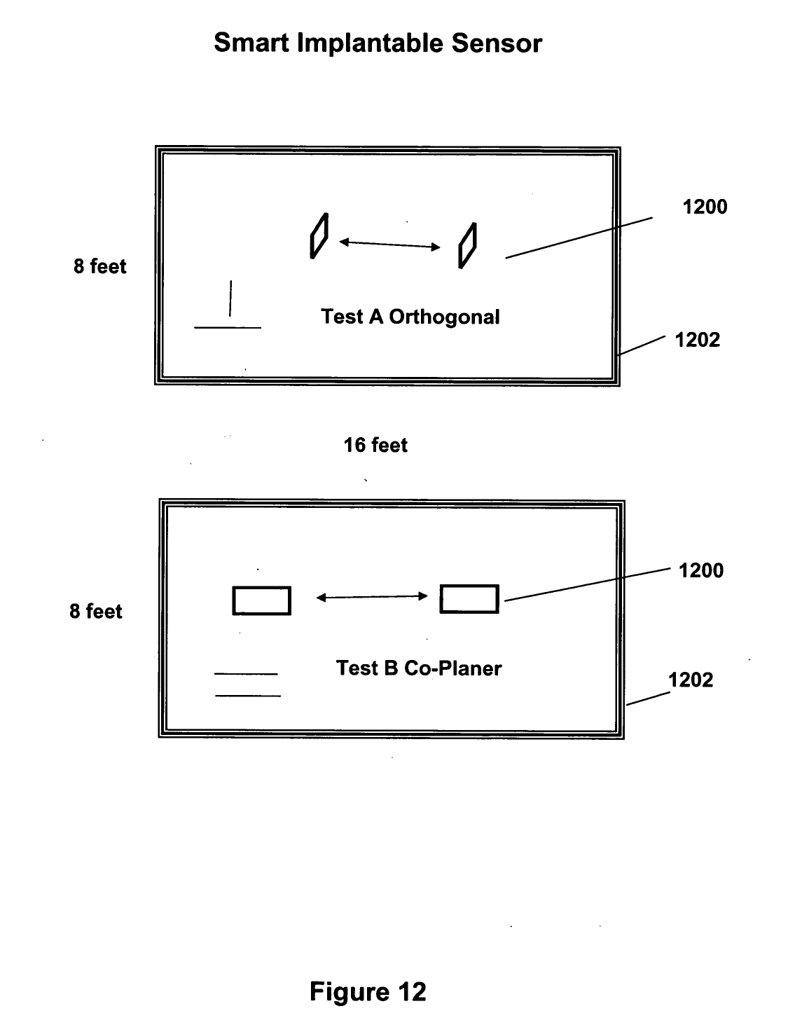

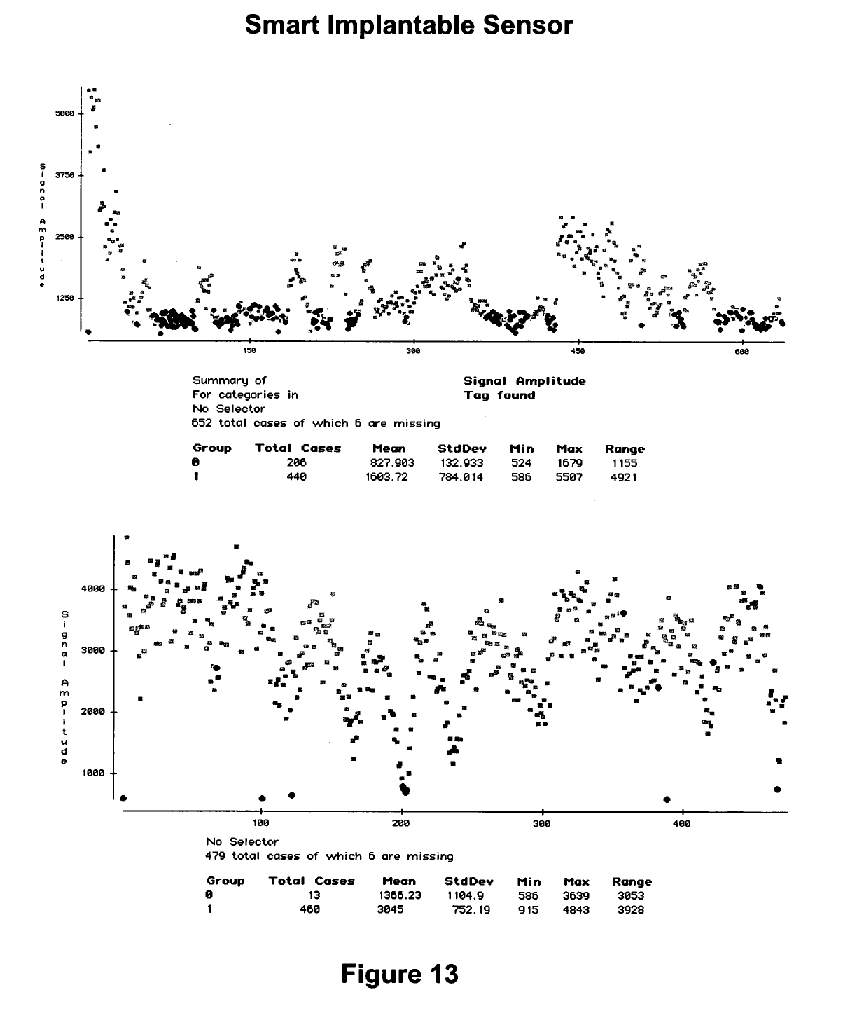

FIG. 12 shows an implant 1200 was held three feet off the floor and two tests were performed. In test A the implant 1200 was held orthogonal (90 degrees) to the floor antenna 1202 and walked around the room randomly. In test B the implant 1200 was held co-planar to the floor antenna 1202. In FIG. 13 raw data shows the signal versus time for test A and B. Dark dots indicate is an error and light dots are correct CRC. It can be seen that in both cases the reads are adequate, even in the non-optimal orientation to read the implant anywhere within the room. Area reads as large as 50′×50′ have been tested with similar results.

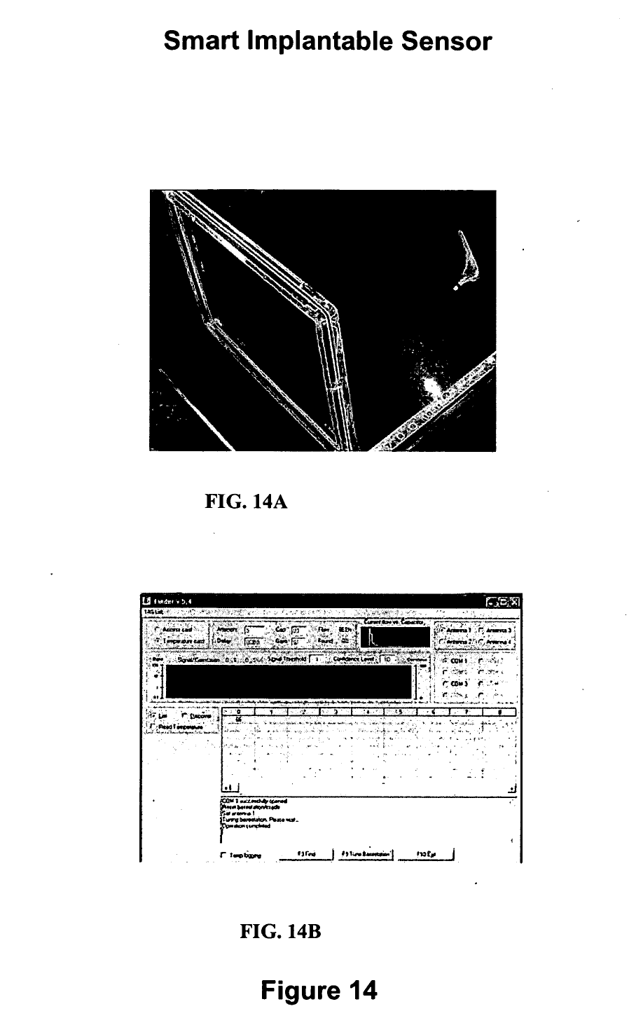

FIGS. 14A and 14B show a test using a coil and reader identical to test in FIG. 9-10. However in this case a steel bone rasp similar to an actual implant was used to test how steel changes the readability of the sensor device. FIG. 14A is a photograph of the basic test stand. A standard HP1217 antenna was placed on a surface with a vertical plane orientation. These antennae normally provide ranges of 10 feet or more with 60N08T-Tags. The modified tag used in this study has a range of about 7 feet. FIG. 14B shows a Software Finder V5.4 screen with options as shown and tuning curve for HP1217.

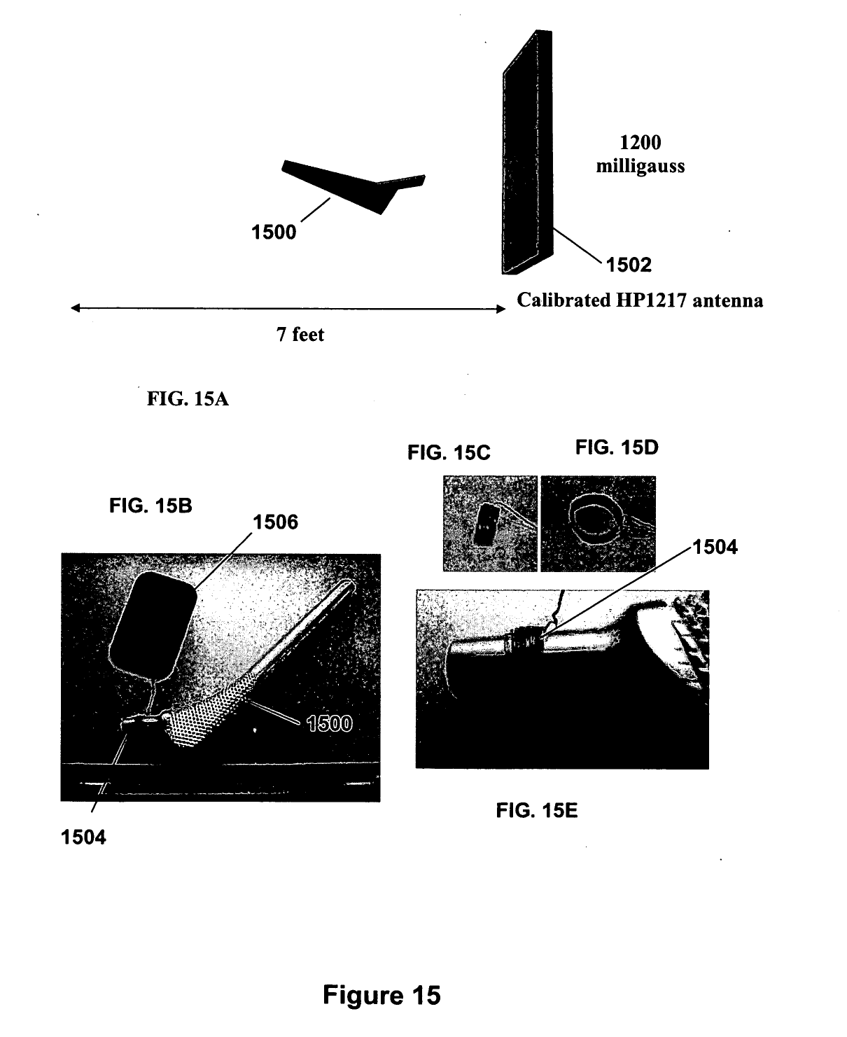

FIGS. 15A-E show the experimental arrangement. FIG. 15A shows bone rasp 1500 and a calibrated antenna 1502. FIG. 15B shoes a bone rasp 1500 with a coil 1504. The coil 1504 is connected to a sensor tag 1506. FIG. 15D shows a close-up view of the rasp 1500 and coil 1504. The implant antenna coil 1504 is about 12 mm in diameter and has been wound around the steel tip of the bone rasp 1500. The same circuit (contained in the black plastic tag) used in other tests was used in this test, however the antenna was tuned with a capacitor. Bone rasp size 18L identical to implantable hip was used as “worst case” test object. The 12 mm coil 1504 was placed over the tapered handle peg. A standard tag 1506 was connected to the coil 1504 for these tests. FIG. 15C shows the 12 mm open coil configuration vertical plane. FIG. 15D shows 12 mm open coil configuration horizontal plane.

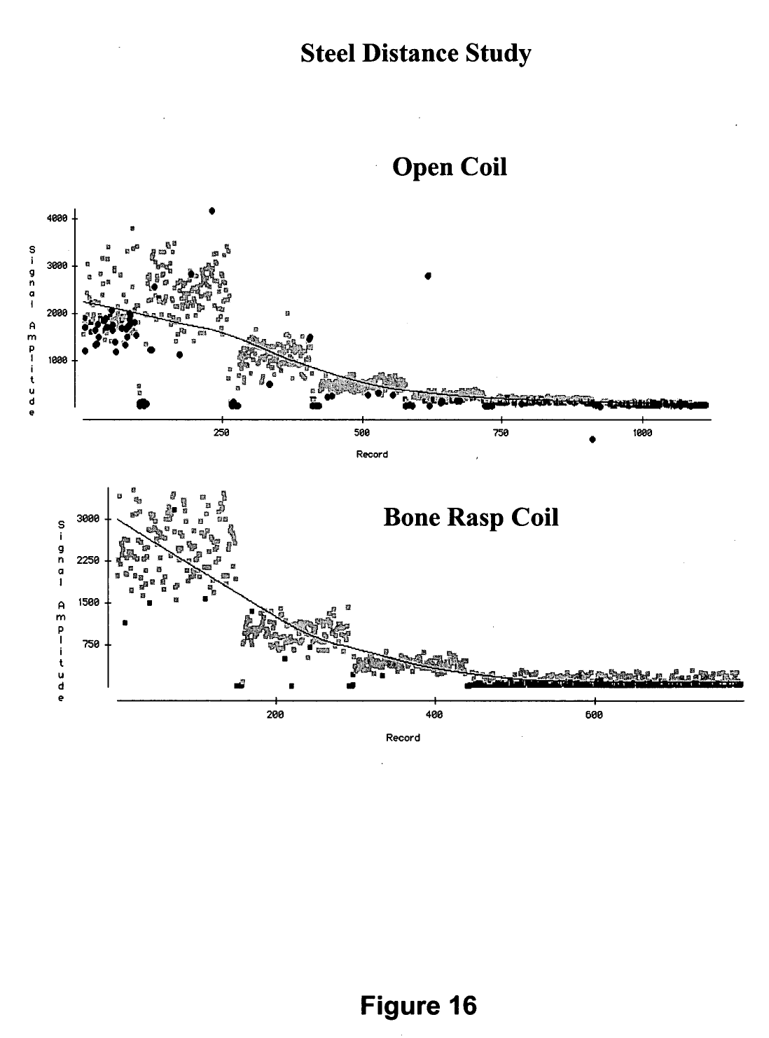

FIG. 16 shows the raw data with an open coil (no steel) and the bone rasp. The steel does decrease the signal and increase the error rate however not sufficiently to change the readability out to about six feet. Raw data for distance study compared the open 12 mm coil and the same coil wrapped around the diameter of the bone rasp. Upper record shows data, light points represent checksum positive no errors, and dark represent missed checksums. Y axis is signal strength and X axis is time (200 msec). The coil was moved in both cases in one foot increments away from the antenna and held at each position for approximately 10 seconds. The errors seen at the start of the open coil data are not meaningful and are related to sync time out errors associated with saturation near the HP1217 antenna. These errors have been corrected in 6033V1.4 tag design. The lower graph is raw data associated with a Rasp with coil wrapped as shown in FIG. 15E.

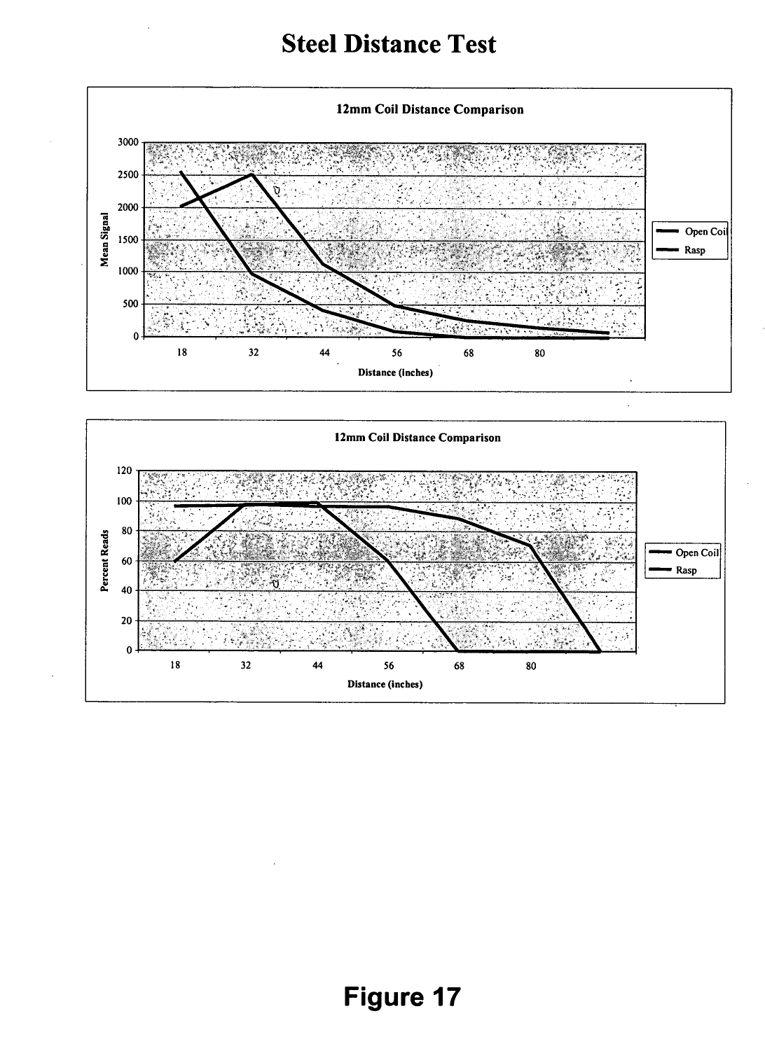

FIG. 17 shows summary graphs showing that the steel does decrease the range by about 20-30% however it is acceptable at near 6 feet from the antenna. The top graph compares mean signal strength vs. distance for the open coil and rasp. The lower graph shows percent error free reads as a function of distance. Forty percent correct reads are acceptable in most applications providing a re-read rate of 5 is used in the system.

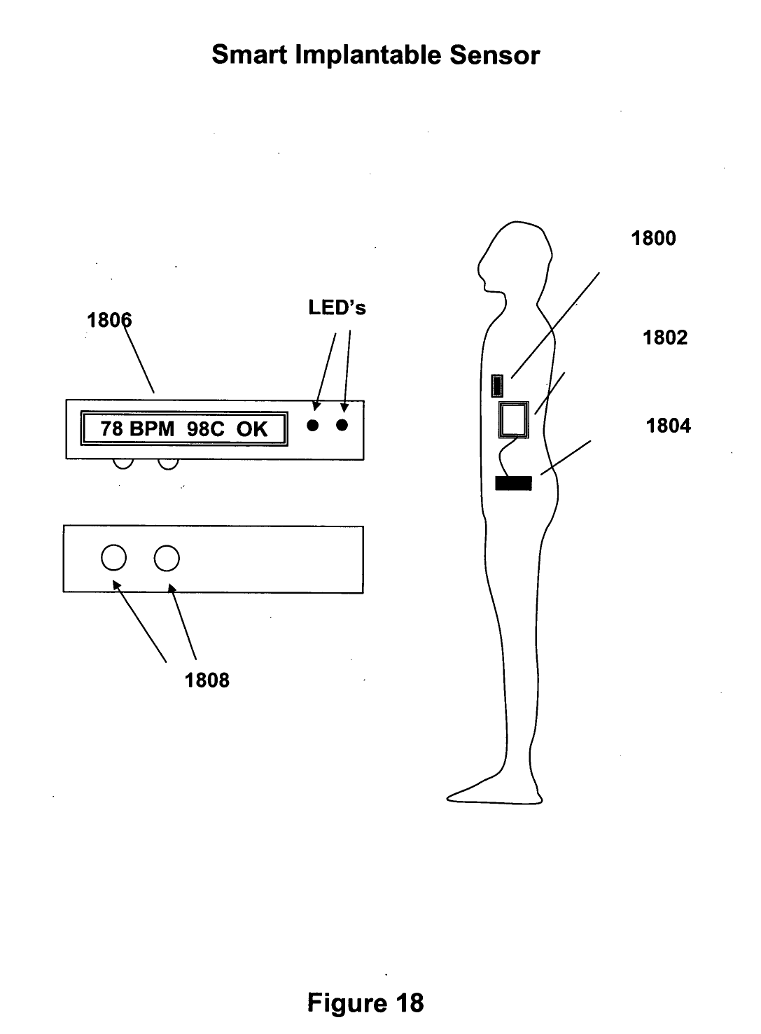

FIG. 18 shows another embodiment for LF radiating transceivers as implantable devices. Here the sensor 1800 is implanted in the upper chest cavity to monitor cardiac rhythms, blood pressure, blood flow, and many biochemicals, such as glucose. An antenna 1802 and a connected external monitor 1804 are on the outside of the patient's body. The top 1806 and side 1808 views of the external monitor are shown.

FIGS. 19-21 are block diagrams of a typical implantable transceiver as described in detail in U.S. Provisional Application 60/652,554, “Ultra Low Frequency Tag and System,” U.S. application Ser. No. 10/820,366, “Damage Alert Tag,” U.S. Patent Provisional Application 60/627,984, “Auditable Authentication of Event Histories, and in U.S. Provisional Application 60/299,727, System and Method for Packaging and Delivering a Temperature-Sensitive Item.

Therefore, while there has been described what is presently considered to be the preferred embodiment, it will be understood by those skilled in the art that other modifications can be made within the spirit of the invention.

We claim:

1. A low frequency implantable sensor and radio tag system, comprising:
a sensor device comprising:
storage storing information including information identifying the device;
an apparatus, coupled to a transceiver, for measuring a body condition for transmission to a reader;
a transceiver, coupled to the storage, the transceiver using induction as a primary communication mode operating at a frequency lower than or equal to 300 kilo Hertz; and
an antenna, coupled to the transceiver, communicating with an external reader.

2. The system of claim 1 further comprising a reader to be worn by a user of the implantable device outside the body of the user.

3. The system of claim 1 wherein the transceiver operates at a frequency sufficiently low to operate near steel.

4. The system of claim 1 wherein the reader comprises a loop antenna to read low frequency signals from the sensor.

5. The system of claim 4 wherein the reader loop antenna reads signals from the sensor when located in the same room as the sensor.

6. The system of claim 2 wherein the reader is connected to a network, and data from the sensor can be captured and maintained at a remote site.

7. The system of claim 6 wherein the network is a wide area network.

8. The system of claim 1 wherein the sensor comprises an apparatus for measuring physical parameters relating to the user.

9. The system of claim 8 wherein the physical parameters include at least one of a number of flexes of a joint, the angle of flexion of the joint, and statistics associated with walking.

10. The system of claim 1 wherein the sensor comprises a glucose detector.

11. The system of claim 1 wherein the sensor comprises a radiation monitor.

12. The system of claim 1 wherein the sensor comprises an electrocardiogram monitor.

13. (canceled)

14. The system of claim 1 wherein the frequency of operation is between 30 and 300 kilo Hertz.
